(19) United States
(12) Patent Application Publication
(10) Pub. No.: US 2013/0041395 A1
(43) Pub. Date: Feb. 14, 2013

(54) INTRODUCTORY ASSEMBLY AND METHOD FOR INSERTING INTRACARDIAC INSTRUMENTS

(71) Applicant: Didier De Canniere, Miami Beach, FL (US)

(72) Inventor: Didier De Canniere, Miami Beach, FL (US)

(21) Appl. No.: 13/442,230

(22) Filed: Apr. 9, 2012

Related U.S. Application Data
(60) Provisional application No. 61/574,798, filed on Aug. 9, 2011.

(51) Int. Cl. A61B 17/34

(52) U.S. Cl. 606/181; 606/185

(57) ABSTRACT

An introduction assembly and method for the insertion of medical instruments through a thoracic passage into a selected one of either the left or right atrium of the heart. Catheters or other instruments dedicated to performing required cardiac maneuvers are passed through an introductory sheath having a distal end disposed within the targeted atrium. Upon completion of the required cardiac maneuvers the instruments are removed from the atrium and a closure assembly is passed through the introductory sheath into a closing relation to an entry site, formed in the pericardium and corresponding atrium wall, to facilitate healing thereof. The introductory assembly and method facilitates the concurrent, operative disposition of a plurality of catheters or other instruments into the interior of the selected atrium through different thoracic passages and entry sites thereby allowing synergistic interaction between the multiple catheters in the performance of the required cardiac maneuvers.

### CLAIM OF PRIORITY

The present application is based on and a claim of priority is made under 35 U.S.C. Section 119(e) to a provisional patent application that is currently pending in the U.S. Patent and Trademark Office, namely, that having Ser. No. 61/574,798 and a filing date of Aug. 9, 2011, and which is incorporated herein by reference.

### BACKGROUND OF THE INVENTION

1. Field of the Invention

The invention relates to intracardiac surgical procedures and more specifically to an assembly and method for introducing medical instrumentation through one or more introductory sheaths, to a predetermined intracardiac depth, into a selected one of the left atrium or right atrium through a thoracic passage and correspondingly disposed intercostal spaces. Upon completion of the required surgical procedure on the interior of the targeted atrium a closure assembly is disposed in closing relation to the entry site for the instrumentation and introductory sheath in the pericardium and targeted atrium wall.

2. Description of the Related Art

When the heart or any of its component parts develops a defect or disease, intracardiac intervention is often necessary to correct, repair, and/or replace damaged or defected cardiac components. Classically, this has been accomplished through surgery in which the chest of the patient is opened and the heart, which is arrested and/or bypassed, is operated on. This can be a very dangerous procedure replete with many possible complications resulting from, at the very least, stopping or bypassing the heart, general anesthesia administered during the procedure, risk of infection from a large opening in the chest cavity, and scarring. Moreover, surgery is not a viable option for many elderly and/or frail patients who are at an increased risk for these complications.

A widely used alternative to cardiac surgery is invasive cardiology, in which catheters are introduced into blood vessels at remote, or peripheral, sites from the heart and are steered through veins and arteries of the body to reach the heart. For example, the femoral vessels, radial artery, subclavian artery and the jugular veins can be used for insertion of catheters for remote cardiac intervention. While this approach avoids many of the risks of surgery, it suffers from significant technical limitations. First, the anatomy and size of the peripheral vessels precludes the use of some catheters. For example, the capillaries and some veins are too narrow to accommodate catheters. Some veins may not be sufficiently sized for a larger catheter, such as in excess of 12 French, or to accept a plurality of catheters simultaneously. The suitability of blood vessels for remote cardiac access may be further exacerbated in many patients, namely the elderly, in which the vessels are narrowed, calcified or tortuous, making access to the heart difficult or impossible. Moreover, the branched network of blood vessels makes the usage of multiple catheters limited to only those catheters having a small caliber. However, even in situations such as these, maneuverability is limited since very little torque can be developed between two catheters threaded through a common blood vessel once inside the heart to address any target structure. This can involve severe limitations since many intracardiac maneuvers require complex access and steering such as, but not limited to, trans-septal punctures, steering the catheter through the inter-atrial septum to access the mitral valve, such as for delivering a MITRACLIP®, percutaneous mitral dilatation, and steering ablation catheters around the openings of the pulmonary veins.

The distance that separates the entry point of the catheter from the target structure is an additional drawback to invasive cardiac measures performed through blood vessels. Moreover, the further the distance from the remote point of entry to the heart, the further the catheter must be threaded and the greater the risk of inadvertently puncturing the wall of a blood vessel, encountering a blockage or collapsed blood vessel, or other obstacle. Moreover, long catheters are also required when the entry point is remote from the heart, necessitating an increase in materials which can become cumbersome to control and maneuver as intended.

More recently, new approaches to intracardiac structures have been introduced to deliver prostheses, such as aortic valves as in the case of transaortic valve implantation (“TAVI”), for patients who do not qualify for a classical surgical replacement and/or whose peripheral vessels are too small to accommodate the large catheters needed to carry the prosthesis. In such an approach, a direct puncture is made in the apex of the left ventricle of the heart via a small incision in the chest wall by an anterior thoracotomy. This approach is becoming more popular and is currently investigated as a route to deliver treatment for other structural heart disease such as, but not limited to auto-implantable mitral prostheses, etc.

However, this entry procedure also has recognized disadvantages. More specifically, this procedure requires general anesthesia and the indicated thoracotomy generates pain, requires long rehabilitation and in known to result in significant complications in especially frail patients. Further, it involves entering the ventricular wall, which leads to a marginal loss of contractile force of the heart, but also a significant risk of bleeding, since the pressure in the ventricle is about 10 times higher than in the atrium. It also requires passage through the ventricular trabeculae and subvalvular mitral apparatus which are needed to prevent backflow of blood during the contraction of the heart, known as systole.

It would therefore be beneficial to implement an improved and proposed introductory assembly and method of accessing the chambers of the heart and performing intracardiac interventions. Such an improved technique would does not require arresting or bypassing the heart and illuminate the use blood vessels for peripheral access to the heart. As a result instrumentation including multiple catheters could be concurrently introduced into predetermined areas of the heart, specifically including the interiors of the right and left atria, in a manner which would eliminate or significantly reduce many of the complications and disadvantages of known surgical procedures.

### SUMMARY OF THE INVENTION

The present invention is directed to an introduction assembly and method for accessing intracardiac structures through the insertion of catheters or other instrumentation into either the right or left atrium. At least one puncture or entry site is formed in the targeted atrium of a beating heart, by inserting a lancet through a thoracic passage by way of an appropriate intercostal space and entering the corresponding portion of the pericardial bag surrounding the targeted atrium of the heart. It is recognized, that in some cases, accessing the atrium through the right side of the chest may be preferred. The introductory assembly and method of the present invention can be used and accomplished from any approach to the heart which enables access to the targeted atrium.

Moreover, the present invention may be used with or without lung deflation, although in some situations it may be preferable to deflate one lung, preferably the right lung, to create additional space in which to work. The present invention also has the distinct advantage of allowing a variety of intracardiac maneuvers to be performed. By way of example such intracardiac maneuvers include, but are not limited to, closing para-valvular prosthetic leaks; closing the left atrial appendage; approaching the mitral and/or tricuspid annuli and/or leaflets to deliver devices that restrain their prolapse or limit their dilatation; encircling the pulmonary veins with ablation lines performed with different energy sources, and repair or replacement of a malfunctioning atrio-ventricular valve. Further, the introductory assembly and method may be utilized surgically after a small, possibly robotically-enhanced right thoracotomy. In this case the atria are opened (“atriotomy”) to manually perform an intracardiac maneuver.

Accordingly, the present invention provides many advantages that overcome the limitations of other known ways of accessing and performing intracardiac interventions. Further by way of non-limiting examples, the practicing of the various preferred embodiments of the present invention reduces the limitations imposed by peripheral access to the heart through blood vessels, such as a narrowing of the vascular tree which precluding catheter passage. The present invention facilitates the ability to insert multiple catheters from different entry points through the thoracic wall and into a targeted atrium. This multiple, concurrent insertion capability thereby permits synergistic action, force, and/or torque between the catheters because they need not be coaxially disposed in relation to each other. This is in contrast to catheters inserted through the venous or arterial vasculature.

In addition, the present invention may be practiced under general anesthesia or sedation, advantageously with temporary single lung ventilation and/or intrapleural carbon dioxide₂ insufflation to temporarily collapse one lung if additional space is needed. The site of the puncture(s) or entry sites may be predetermined with imaging, such as 3D CT reconstruction of the cardiac structures relative to the rib cage, and may be performed in a cath lab or preferably a hybrid operating room under fluoroscopy, preferably with transoesophageal echographic guidance.

In more specific terms, the present invention includes an introduction assembly for the insertion of medical instruments such as, but not limited to, catheters through a thoracic passage and into either the right or left atrium of the heart. As such, a puncturing instrument is dimensioned and structured to form an entry site into the targeted right or left atrium by first penetrating a corresponding portion of the pericardial bag. The puncturing instrument is introduced through a thoracic passage and an appropriate intercostal space. In addition, an elongated introductory sheath or like tubular structure includes a central lumen and is movably disposed over the puncturing instrument so as to extend through the entry site formed in both the pericardial bag and the targeted right or left atrium. The sheath also includes a distal end having a predetermined intracardiac length which is positioned on the interior of the targeted atrium.

Additional structural features of the inserted introductory sheath include a buffer disposed thereon in segregating relation between the distal end of the sheath, which enters the targeted atrium, and the remainder of the sheath disposed exteriorly of the targeted atrium. As applied, the buffer is disposed in confronting disposition with an exterior portion of the pericardial bag, which corresponds to the entry site. As such the buffer is determinative of the intracardiac length by its spacing from the extremity of the distal end, which is disposed into the targeted atrium through the entry site. The central lumen of the introductory sheath is dimensioned and configured to receive and facilitate passage therethrough of instrumentation, such as catheters, which are dedicated to the performance of predetermined cardiac maneuvers within the targeted atrium. Subsequent to the completion of the intended cardiac maneuvers within the selected atrium, a closure assembly is disposable in an operative position in closing relation to the entry site formed in both the pericardial bag and the atrium wall of the targeted atrium.

In one or more preferred embodiments, the buffer comprises or is directly associated with a securing assembly which includes a source of vacuum or negative pressure. The buffer is connected to the vacuum source preferably through flow lines, conduits or other appropriate structures connected to or mounted on the introductory sheath. As such, fluid communication is established between the buffer and the vacuum source to the extent that and appropriate negative pressure is developed and communicated to the buffer through the flow lines. The negative pressure is sufficient to removably secure the buffer to the exterior surface of the pericardial bag immediately adjacent to the entry site formed in both the pericardial bag and the atrium wall.

Yet additional structural and operative features of at least one preferred embodiment of the buffer include it having a collapsible construction. Moreover, the collapsible construction of the buffer may be at least partially defined by a plurality of pads extending outwardly from the exterior of the sheath into a disposition which facilitates the aforementioned removable securement to the exterior of the pericardial bag adjacent to the entry site. In yet another preferred embodiment, the buffer may include an annular configuration connected to and at least partially surrounding exterior portions of the sheath. As such, the buffer is extendable transversely outward from the sheath into the aforementioned removable securement. Therefore, by the application of the negative pressure or vacuum associated with the buffer, the introductory sheath is disposed in movement restricting relative to the entry site. As should be noted, the regulation of fluid flow between the vacuum source and the buffer will allow control over the attachment of detachment of the buffer from its stabilized position relative to the pericardial bag.

Yet additional structural and operative features of at least some of the preferred embodiments of the present invention include the aforementioned closure assembly. More specifically, the closure assembly may comprise a first segment and a second segment respectively and concurrently disposable interiorly and exteriorly of the entry site. As such, the first segment of the closure assembly passes through the lumen of the introductory sheath, through the entry site and into the interior of the targeted atrium. Cooperatively, the second segment of the closure assembly also passes through the lumen of the introductory sheath and is disposed exteriorly of the pericardial wall and entry site. Interconnecting structure between the first and second segments of the closure assembly may be operatively manipulated such as from an exterior of the proximal end of the introductory sheath. Such manipulation of the interconnecting structure will bring the first and second segments to closing relation to the entry site as they are respectively disposed on the interior of the targeted atrium and on the exterior of the pericardial wall. When disposed in the intended sealing relation to the entry site, the first and second segments will effectively “sandwich” the entry site therebetween and facilitate its closure.

The segments of the closure assembly may be formed of a material which will dissolve within the time required for the healing of the entry site. Moreover, the first and second segments of the closure assembly are also formed of a collapsible material which has an at least minimal inherent bias. These collapsible characteristics allow the folding or size reduction of the first and second segments as they pass through the introductory sheath to the entry site. However, upon passage from the open end of the lumen of the introductory sheath, each of the first and second segments will be automatically expanded into an intended operative size and configuration for their respective disposition into closing relation to the entry site.

As set forth above, the various preferred embodiments of the present invention are directed not only to the introduction assembly, as generally set forth above, but also to a method of introducing medical instrumentation through a thoracic passage and into a targeted one of the either the right or left atrium of the heart. Accordingly, in cooperation with the introductory assembly as set forth above, the method of at least one preferred embodiment of the present invention comprises the forming of at least one entry site into the targeted atrium and into a corresponding part of the pericardial wall. The aforementioned introductory sheath is positioned such that a distal end thereof, having the predetermined intracardiac length, extends through the thoracic passage and the entry site into the targeted atrium along a predetermined length. Once so positioned, appropriate instrumentation, such as catheters, dedicated to perform the intended predetermined cardiac maneuvers, are passed along the interior of the introductory sheath and into the targeted atrium through the entry site. Once the predetermined cardiac maneuvers have been completed the instrumentation is removed from the selected atrium through the introductory sheath. Thereafter the aforementioned closure assembly is passed through the central lumen of the introductory sheath and into a closing relation with the entry site.

As also set forth above, one of the distinct advantages of the present invention is the ability to concurrently insert multiple catheters into the targeted atrium so as to enable the interaction between the concurrently present instruments within the selected atrium. Accordingly, one or more preferred embodiments of the method of the present invention comprises forming a plurality of different entry sites into the targeted atrium and corresponding pericardial wall and positioning different introductory sheaths through the correspondingly positioned ones of a plurality of entry sites. In addition, the corresponding distal ends of the plurality of the introductory sheaths have appropriate intracaridac lengths so as to facilitate the maneuverability and manipulation of the instrumentation once present in the targeted atrium. Upon completion of the required cardiac maneuvers within the selected atrium, a plurality of closure assemblies will pass through different ones of the plurality of introductory sheaths so as to operatively dispose the first and second segments of each of the closure assemblies in closing relation to the formed entry sites, as set forth above.

Accordingly, the present invention overcomes the disadvantages and problems associated with known surgical techniques by implementing the various preferred embodiments of the subject introductory assembly and method for the insertion of instrumentation through a thoracic passage into a selected one of the right or left atrium, as will be described in greater detail hereinafter.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic representation of the heart including the implementation of the present invention including the introduction of a plurality of medical instruments into a selected one of the right or left atrium of the heart.

FIG. 2A is a schematic representation of the anatomy of the heart as seen from the right chest.

FIG. 2B is a schematic representation of the anatomy of the heart as seen from the right chest and including schematic designations of surgical sites for cardiac maneuvers using the introductory assembly and method of the present invention.

FIG. 3 is a front view in partial cutaway of one preferred embodiment of the introductory assembly of the present invention.

FIG. 4 is a front view of the embodiment of FIG. 3 representing a successive step in the method of implementing the introductory assembly of the present invention.

FIG. 5 is another preferred embodiment of the introductory assembly of the present invention similar to but distinguishable from the embodiment of FIGS. 3 and 4.

FIG. 6 is a front view of the embodiment of FIG. 5 in a successive step of the method of implementing the introductory assembly of the present invention.

FIG. 7A an exterior perspective view of the embodiment of FIGS. 5 and 6.

FIG. 7B is an end view of the embodiment of FIG. 7A.

FIG. 8A is a front perspective view of yet another preferred embodiment similar to but distinguishable from the embodiment of FIGS. 7A and 7B.

FIG. 8B is an end view of the embodiment of FIG. 8A in partial phantom.

FIG. 9 is front view in partial cutaway of the method of implementing the introductory assembly of the embodiment of FIGS. 5 and 6.

FIG. 10 is a front view of the representing the method of implementing the introductory assembly of the embodiment of FIG. 9.

FIG. 11 is a front view representing an additional step of the method of implementing the introductory assembly of the embodiment of FIGS. 9 and 10.

Like reference numerals refer to like parts throughout the several views of the drawings.

### DETAILED DESCRIPTION OF THE PREFERRED EMBODIMENT

As represented in the accompanying Figures, the present invention is directed to an introduction assembly and attendant method for the insertion of medical instruments, such as catheters, through a thoracic passage and corresponding intercostal spaces into either a right or left atrium of the heart for the purpose of performing predetermined cardiac maneuvers on intracardiac structures, as required.

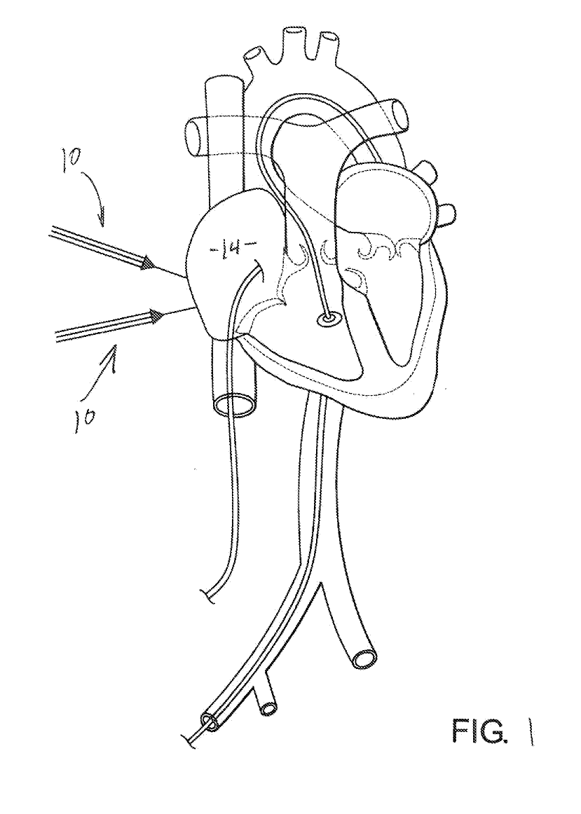

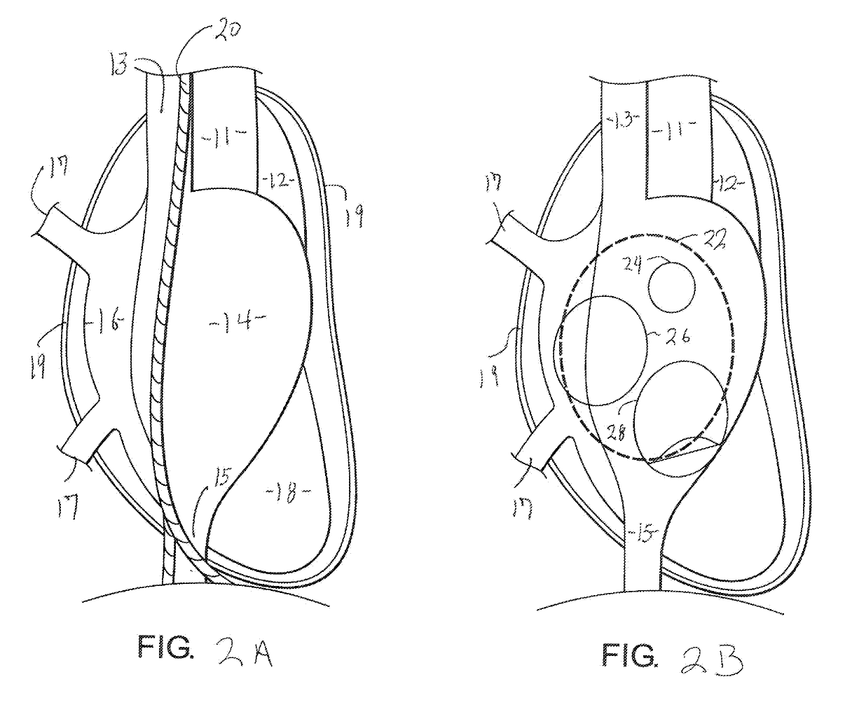

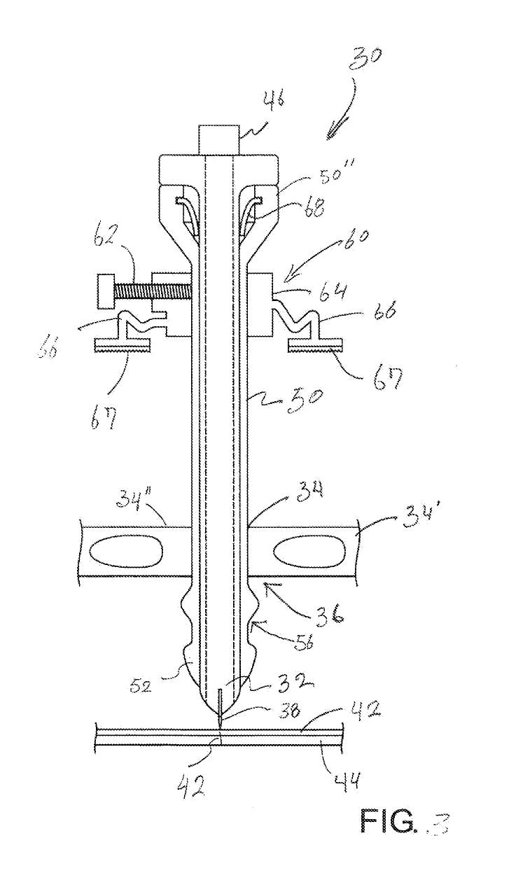

For purposes of clarity and reference, FIGS. 1, 2A and 2B are schematic representation of the anatomy of the heart. Accordingly, implementing one or more preferred embodiments of the present invention, multiple instruments, including catheters generally indicated as 10, may be concurrently disposed in either the right or left atrium of the heart. As will be set forth in greater detail hereinafter, the instruments 10 pass through the thoracic wall and appropriate ones of intercostal spaces into an interior of a targeted one of the left or right atrium by means of a formed entry site in the pericardium and selected atrium wall. In addition, FIG. 1 presents known or substantially conventional surgical techniques in which catheters are introduced into blood vessels at remote or peripheral sites from the heart and are steered through veins or arteries of the body to reach the heart.

By way of example, the femoral vessels, radial artery, subclavian artery and the jugular veins can be used for the insertion of catheters for remote cardiac intervention. As is well recognized, this peripheral approach avoids many of the risks of open heart surgery but it suffers from significant technical limitations at least partially based on the anatomy and size of the peripheral vessels or a condition existing in some patients resulting in the narrowing or calcification or torturous configuration thereof, making access to the heart difficult, as generally set forth above.

With primary reference to FIGS. 2A and 2B schematic representations of the anatomy of the heart, as seen when viewing the right chest, includes the aorta 11, pulmonary artery 12, superior vena cava 13, right atrium 14 and inferior vena cava 15. Additional representations include the pulmonary veins 17 as well as the right ventricle 18, the pericardial bag 19 and the pulmonary veins 20. For purposes of further reference, FIG. 2B provides a schematic representation of the various surgical sites in which possible cardiac maneuvers may be performed using the assembly and method of the present invention. More, specifically, the perimeter 22 generally defines the zone or area wherein multiple instruments may be concurrently introduced into the right atrium 14 through different thoracic passages and corresponding entry sites by implementing the various preferred embodiments of the present invention. Additional schematic representations include the projection of the left artial appendage 24; the projection of the mitral valve annulus 26 and the projection of the tricuspid valve annulus 28.

Therefore, required cardiac maneuvering of multiple catheters and other instruments can be individually and cooperatively maneuvered in the indicated surgical sites or zones by implementing the assembly and method, as described in greater detail with reference to FIGS. 3 through 11.

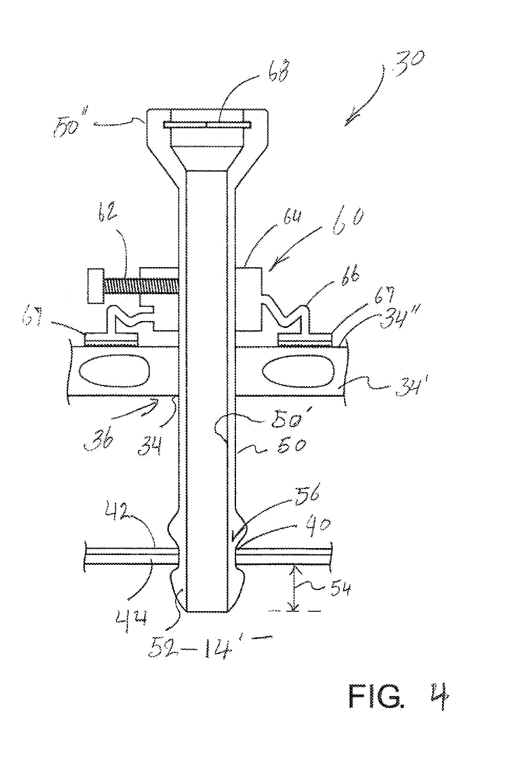

With initial reference to FIGS. 3 and 4, one preferred embodiment of the introductory assembly is generally indicated as 30. More specifically, a puncturing instrument 32, which may be in the form of a puncturing needle, lancet, etc. is utilized to form a thoracic passage 34 in the thoracic wall 34′ through an intercostal space 36 between appropriately positioned ribs, as schematically represented. Further, the lancet 32 may have a puncturing or cutting blade 38 of sufficient structure to form an entry site 40 extending through both the wall 42 of the pericardial bag and the corresponding disposed part of the wall 44 of the selected or targeted atrium 14.

While the puncturing instrument or lancet 32 may vary in construction and operation, one embodiment thereof includes the cutting blade 38 selectively disposable between an outwardly extended, operative position, as represented in FIG. 3, or an inwardly disposed retracted position, not shown for purposes or clarity. In order to accomplish this selective positioning of the blade 38, an accessible positioning member or like structure 46 is connected to the blade 38 and may be mounted on the lancet 32 at generally a proximate end thereof. As such, the positioning member 46 is disposed exteriorly of the thoracic wall 34′ and is thereby readily accessible for manipulation by medical personnel to accomplish the extension or retraction of the blade 38, as required. With further reference to FIGS. 3 and 4, an introductory sheath 50 includes a central channel or lumen 50′ facilitating the coaxial alignment and overlying, covering relation of the sheath 50 relative to the puncturing instrument 42. Once the entry site 40 is formed, the distal end 52 of the introductory sheath 50 passes there through. As a result, the central lumen 50′ of the introductory sheath 50 is disposed in accessible communication with the interior 14′ of the selected atrium 14, as generally indicated in FIG. 4.

The passage and positioning of the distal end 52 of the sheath 50 is controlled and/or restricted through the provision of a buffer, generally indicated as 56. As will be apparent from additional description provided hereinafter, the buffer 56 may be defined by a variety of different structures. However, in each of the possible structural modifications, the buffer 56 is disposed and configured to limit or restrict the length of the distal end 52 which passes into the interior 14′ of the selected or targeted atrium. More specifically, the disposition and structural features of the buffer 56 will determine an “intracardiac length” 54 of distal end 52 which defines the length of the distal end 52 allowed to be inserted within the interior 14′ of the selected atrium. While the intracardiac length 54 may vary, the conventional length would be generally from about 1.5 cm to 2 cm. The intracardiac length 54 is sufficient to facilitate entry of intended instruments into the atrium but is at least partially restricted to facilitate manipulation and maneuvering of a catheter or other instrument passing through the introductory sheath 50 into the interior 14′ of the targeted atrium. As a result, required cardiac manipulation of intracardiac structure intended for treatment, repair, replacement, etc. may be more efficiently accomplished.

Additional structural and operative features of the introductory assembly 30 include a stabilizing assembly 60 adjustably and/or movably connected to the introductory sheath 50. The stabilizing assembly 60 is selectively positioned relative to the exterior of sheath 50 into and or out of engagement with the exterior surface 34″ of the thoracic wall 34′. Moreover, the structural and operative features of the stabilizing assembly are such as to maintain a preferred and/or predetermined angular orientation of the sheath 50 relative to the thoracic wall 34′ as the sheath 50 passes through the thoracic passage 34 and the entry site 40. While the schematic representations of FIGS. 4-6 and 9-11 show a substantially perpendicular or direct inline relation between the axis of the sheath 50 and the thoracic wall 34′, FIG. 1 more accurately indicates that the various instruments 10 may assume a variety of different angles as they extend through the thoracic wall into the selected atrium. Therefore, the stabilizing assembly 60 includes a lock or like fixing member 62 movable relative to a base 64 into a removable locking engagement with the exterior of the introductory sheath 50. In addition, adjustable legs or like members 66 have engaging pads 67 structured to resist or restrict relative movement between the exterior of the pad 67 and the exterior surface 34″ or the thoracic wall 34′ to which the stabilizing assembly 60 is removably secured. As a result, the stabilizing assembly 60 facilitates the maintenance of the sheath 50 and instruments passing there through at a preferred predetermined angular orientation relative to the thoracic wall 34′.

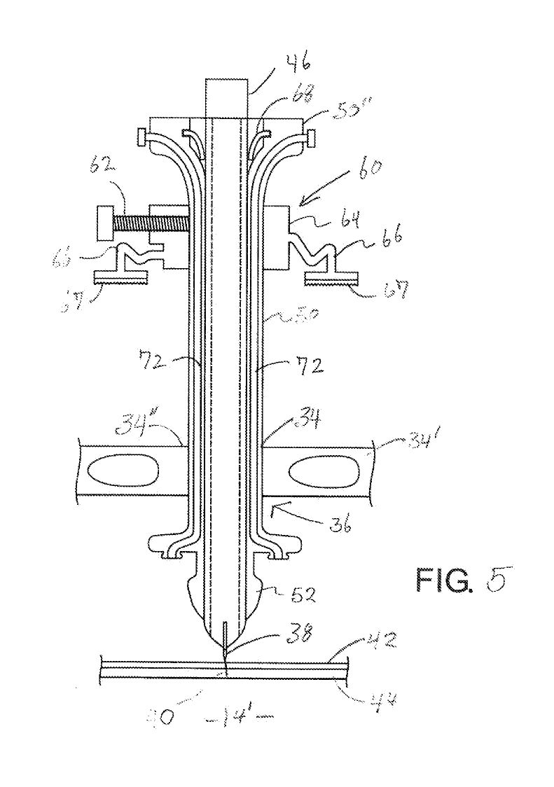

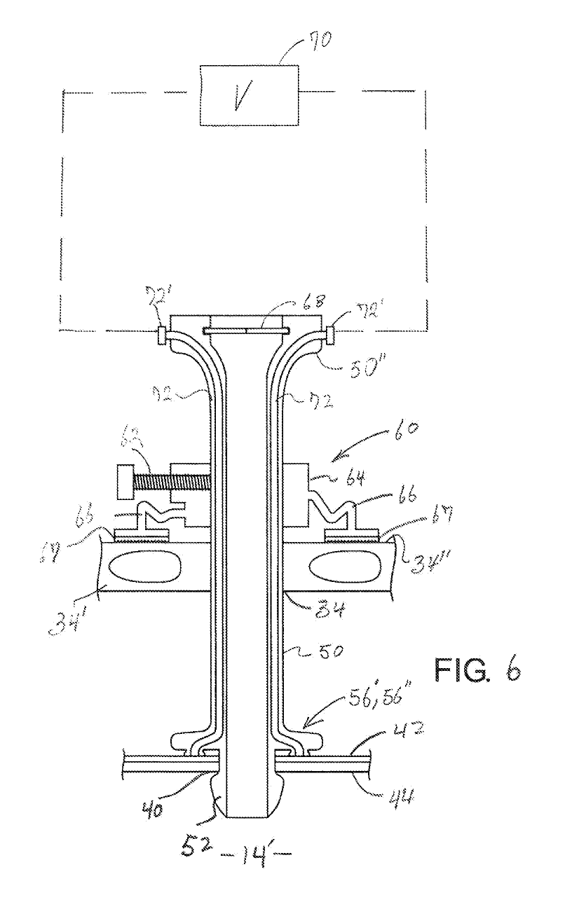

As also indicated in one or more of the various preferred embodiments of introductory sheath 50, a valve structure generally indicated as 68 is connected at or adjacent to the proximal end 50″. More specifically, the valve structure 68 is disposed within a portion of the interior lumen 50′ and is structured to facilitate the passage of instruments into and through the lumen 50′ as they are introduced into the open proximal end 50″, as clearly represented in FIGS. 5 and 9 through 11. However, the valve structure 68 will automatically close absent the existence of instrumentation within the interior lumen. In its closed orientation, as represented in FIGS. 4 and 6, the valve structure is operatively disposed to prevent back bleeding and/or air embolism and while enabling the sequential introduction of dedicated catheters to perform the intracardiac maneuvers.

Therefore, the valve structure 68 may be considered, but is not limited to, a one way valve structure which may include an inherent bias or other operative structure which facilitates its closure into fluid sealing relation to the interior lumen 50′ absent the presence of instrumentation within the lumen 50′.

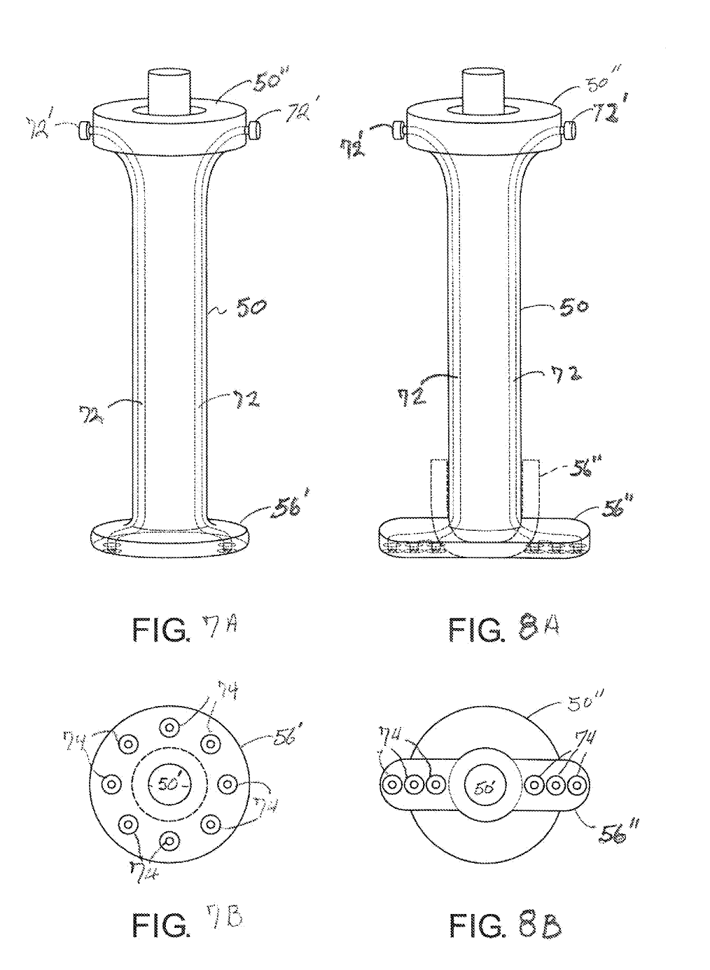

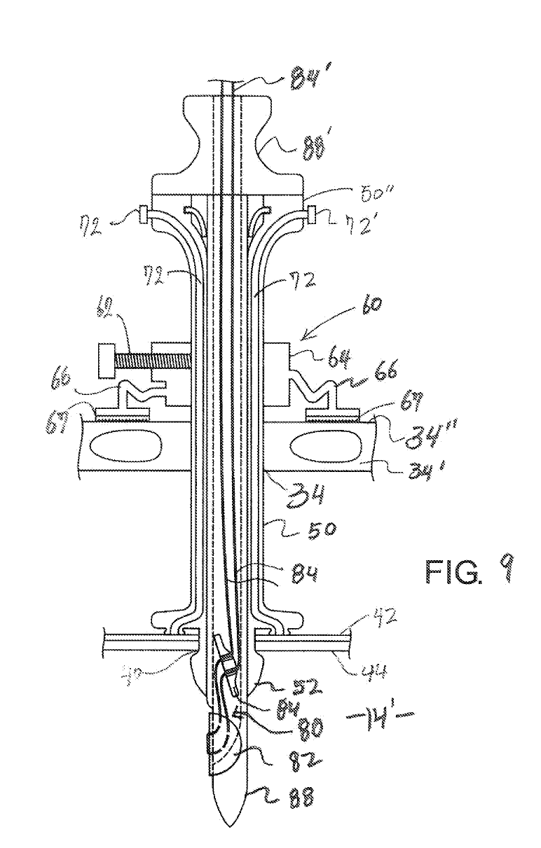

As represented in FIGS. 5 through 11 yet another preferred embodiment of the present invention comprises structural modifications of the buffers, generally indicated as 56′ and 56″. The structural and operative differences are described in greater detail with primary regard to FIGS. 7A, 7B, and 8A, 8B. More specifically, each of the buffers 56′ and 56″ is secured to the exterior of the pericardial bag 42 by means of vacuum or negative pressure generated by a vacuum source generally indicated as 70. The vacuum source 70 is connected in fluid communication to the buffers 56′, 56″ by means of appropriate conduits 72 or other interconnecting flow communicating structure. As such, the flow communicating structures or conduits 72 may be mounted on or at partially within the introductory sheath 50. As selectively operated, the vacuum source 70 may produce a negative pressure on or with the buffer structure 56′, 56″ which in turn is exerted on the exterior surface of the pericardial bag 42. As a result, the buffers 56′ and 56″ will be maintained in a secure, stable but removable engagement with the exterior of the pericardial bag 42. Such a removable securement will further facilitate the stable, intended positioning of the distal end 52 within the interior 14′ of the targeted atrium.

As should be apparent, control or regulation of the negative pressure exerted by the buffer 56′, 56″ on the pericardium 42 may be regulated by the operation of the vacuum source 70. Therefore, when activated sufficient negative pressure is exerted on the exterior surface of the pericardium 42 by the buffer 56′, 56″ in order to maintain the buffer 56′, 56″ in secure engagement therewith. However, by diminishing or eliminating the negative pressure, by regulating the operation of the vacuum source 70, a detachment of the buffer 56′, 56″ as well as the introductory sheath 50 from the entry site 40, as represented in FIG. 11, can be easily accomplished. Additional structural features associated with FIGS. 6 through 11 include the vacuum or negative pressure source 70 being removably connected to the proximal end 50″ of the introductory sheath 50 by appropriate connectors 72′ attached to or associated with the fluid flow conduits 72.

With primary reference to the embodiment of FIGS. 7A and 7B, the buffer 56′ comprises a substantially annular configuration including at least one but more practically a plurality of openings 74 formed in the under surface thereof. As should be apparent, the openings 74 are disposed in direct fluid communication with the exterior surface of the corresponding pericardial bag 42 as represented in FIGS. 6 and 9-10 and thereby exert the aforementioned negative pressure on the pericardial bag 42. As set forth above, the negative pressure is sufficient to maintain a secure engagement of the buffer 56′ with the exterior surface of the pericardial bag 42 thereby maintaining the stability and accurate disposition of the introductory sheath 50.

With primary reference to FIGS. 8A and 8B, yet another embodiment of the buffer 56″ is represented which includes at least one but preferably a plurality of outwardly extending pads 57. Each of the pads 57 is disposed in fluid communication with the vacuum source 70 through the aforementioned conduits or like flow communicating structures 72. Somewhat similar to the embodiment of FIGS. 7A and 7B, the pads 57, defining the buffer 56″, also include a plurality of opening 74 which are disposed in confronting engagement of the exterior surface of the pericardium 42 and thereby exert a suction or negative pressure thereon. The exerted negative pressure is sufficient to maintain the buffer 56″ into a stable but removable connection with the pericardial bag 42 substantially adjacent the entry site 40. Additional structural features of the buffer 56″ include its ability to be selectively disposed in a collapsed or retracted orientation as represented in phantom lines in FIG. 8A. As should be apparent, when in the collapsed position, the pads 57 of the buffer 56″ take up less room thereby facilitating the positioning thereof into the intended operative position as they are disposed through the thoracic passage 34 of the thoracic wall 34′.

Further, the positioning or orientation of the pads 57 in the operative position may be at least partially “automatic” by structuring the pads from a material which has at least a minimal inherent bias. Once the buffer 56″ is disposed in confronting and/or adjacent relation to exterior surface of the pericardium 42 the inherent bias of the material from which the pads 57 are formed will facilitate their “automatic” outward orientation into the operative position of FIGS. 8A and 8B.

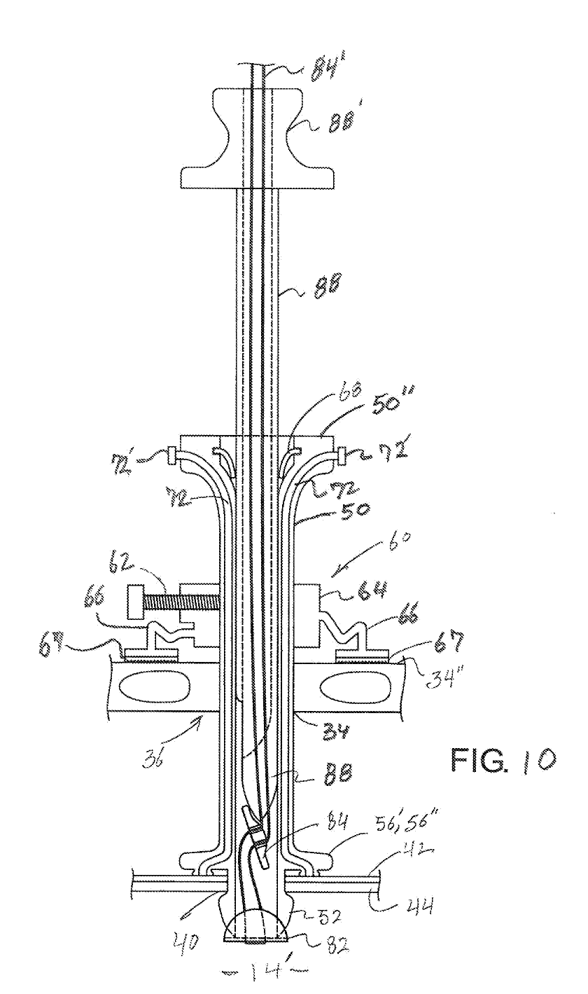

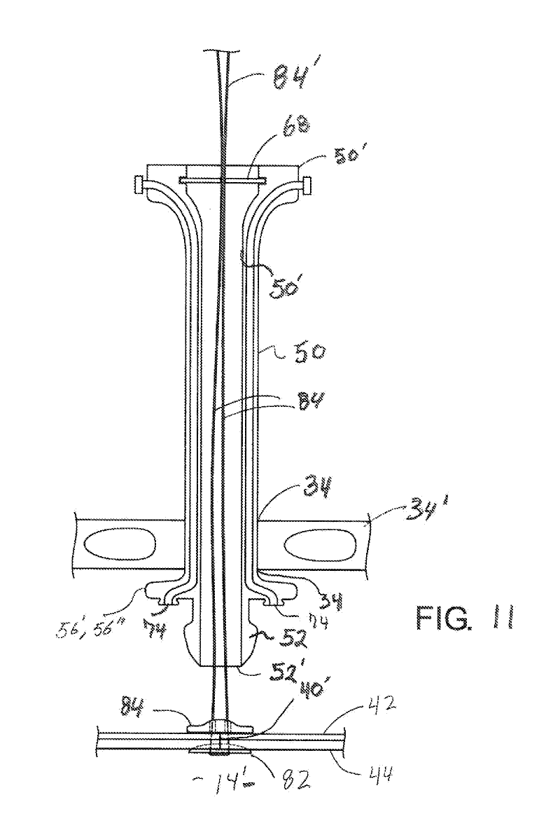

Yet another embodiment of the present invention is represented in FIGS. 9 through 11 and is related to a closure assembly generally indicated as 80. However, it is emphasized, that the closure assembly 80, while specifically represented for use with the embodiments of FIGS. 5 through 11 is also operatively structured for use with the embodiments of FIGS. 3 and 4 as described above. Therefore, the closure assembly 80 is selectively disposable within the lumen 50′ of the introductory sheath 50 and for positioning in closing or sealing relation to the entry site 40 An operative positioning of the closure assembly 80 is accomplished upon a removal of the distal end 52 from the interior 14′ of the selected atrium, as represented in FIG. 11. For purposes of clarity the closed or sealed entry site is represented in FIG. 11 as 40′. Moreover, the closure assembly 80 includes a first segment 82 and a second segment 84 at least initially disposed in separated relation to one another. However, in at least one preferred embodiment of the closure assembly 80 includes an interconnecting structure, such as a cord or like structure 84, which may be manipulated interconnect the first and second segments 82 and 84 into the closing relation to the entry site 40′. As such, the interconnecting structure 84 extends through substantially the entire length of the lumen 50′ and includes a portion 84′ which is assessable from the exterior of the introductory sheath 50, as clearly indicated. As implemented, the first segment 82 passes into the interior 14′ of the selected atrium through the open entry site 40 formed in the pericardium 42 and the atrium wall 44. Such interior positioning of the first segment 82 may be accomplished by appropriate instrumentation 88 which also may be in the form of a positioning catheter or like structure. The instrumentation 88 also passes through the interior lumen 50′ of the introductory sheath 50 and includes a positioning member 88′ protruding outwardly from the open proximal end 50″ of the introductory sheath 50 as represented in FIGS. 9 through 11. With primary reference to FIG. 10, once the first segment is disposed on the interior 14′ of the selected atrium, the second segment 84 is disposed or remains within the interior lumen 50′ adjacent to the distal end 52. Once the first segment 82 is disposed on the interior 14′ of the atrium, the distal end 52 of the introductory sheath 50 is removed from the interior 14′ of the selected atrium and passes back through the open entry site 40 along with the second segment 84 remaining on the interior of the lumen 50′.

Subsequent to the removal of the distal end 52 of the introductory sheath 50 from the entry site 40 and upon closure of the entry site 40, as at 40′, the positioning instrument 88 will serve to remove the second segment 84 from the interior lumen 50′ through the opening 52′ of the distal end 52. Appropriate manipulation of the exterior, accessible end 84′ of the interconnecting structure 84 will then serve to dispose both the first segment 82 and the second segment 84 into the closing relation to the now closed entry site 40′ as clearly represented in FIG. 11. When the operative closing relation as represented in FIG. 11, the first closing segment 82 will be disposed in confronting engagement with the interior surface of the selected or targeted atrium wall 44. In cooperation therewith, the second exterior closing segment 84 will be disposed in confronting engagement with the exterior surface of the pericardium 42. As such the closed entry site 40′ will thereby be effectively “sandwiched” therebetween to prevent leakage or passage of fluid therethrough. This closing sealing relation of the closing assembly 84, relative to the closed entry site 40′, will facilitate the healing thereof.

Additional features of the closure assembly 80 and specifically including the first and second closing segments 82 and 84 are their formation from a material which has an at least minimal inherent bias. As such, both the first and second closing segments 82 and 84 may be disposed in at least partially folded or otherwise collapsed orientation as they pass through the interior lumen 50′ of the introductory sheath 50. However, once passing out of the opening 52′ of the distal end 52, the “inherent bias” of the material of the first and second closing segments 82 and 84 will facilitate their “automatic” expansion into the operative position clearly represented in FIG. 11. Also of note is the forming of the first and second closing segments 82 and 84 from a material that will eventually dissolve on a timely basis by the exposure to ambient bodily fluids. The time in which the first and second closing segments 82 and 84 will be dissolved effectively coincides to the healing of the closed entry site 40′.

Accordingly, the introduction assembly and method for the insertion of medical instrumentation through a thoracic passage into a targeted atrium of the heart overcomes many of the disadvantages and complications associated with conventional or known related surgical procedures, as set forth above.

By implementing one or more of the embodiments of FIGS. 3 through 11, the attendant method comprises forming at least one, but if required, a plurality of entry sites 40 into a targeted atrium 14 and positioning different introductory sheaths 50 through different thoracic passages 34 and corresponding ones of the formed entry sites 40. The distal end 52 of each of the introductory sheaths 50 is inserted through corresponding entry sites 40 into the interior 14′ of the selected atrium 14 to a depth corresponding to the intracardiac length 54 of the inserted distal end 52. Once the one or more sheaths 50 are inserted through respective ones of the entry sites 40, catheters or other instruments dedicated to perform predetermined cardiac maneuvers pass through the one or more introductory sheaths 50 into the targeted atrium 14 through the corresponding entry sites 40. Thereafter and upon completion of the required cardiac maneuvers, the inserted catheters or instruments are removed from the interior 14′ of the targeted atrium 14 back through the central lumen 50′ of the respective introductory sheaths 50.

In order to close or seal the entry sites 40 a plurality of closure assemblies 80 are passed through the interior lumen 50′ of each of the one or more introductory sheaths 50. In establishing a closing relation of the closing assemblies 80 with the entry sites 40, a first closing segment 82 and a second closure segment 84 of each closure assembly 80 are respectively disposed interiorly and exteriorly of the entry site 40. As such, the entry sites 40, or 40′ when closed, are disposed in a substantially “sandwiched” relation between the corresponding first and second closure segments 82 and 84. After operative positioning of the closure assemblies 80, each of the one or more introductory sheaths 50 are removed from the operating field by movement back through the respective thoracic passages 34.

Accordingly, the introduction assembly and method of the present invention for the insertion of medical instruments through a thoracic passage into a targeted atrium of the heart are believed to overcome many of the disadvantages and complications associated with conventional or known related surgical procedures, as set forth above.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. An introduction assembly for the insertion of medical instruments through a thoracic passage and into a targeted atrium of the heart, said introduction assembly comprising:
a puncturing instrument dimensioned and structured to form an entry site into the targeted atrium and corresponding pericardial bag,
an elongated sheath including a central lumen movably disposed over said puncturing instrument through the entry site,
said sheath including a distal end having a predetermined intracardiac length,
a buffer disposed on said sheath in segregating relation between said distal end and a remainder of said sheath,
said buffer structured to limit insertion of said distal end into the targeted atrium through the entry site,
a closure assembly disposable in an operative position in closing relation to the entry site in the targeted atrium subsequent to removal of said sheath therefrom, and
said sheath and central lumen dimensioned and configured to receive and facilitate passage therethrough of instrumentation dedicated to perform predetermined cardiac maneuvers.

2. An introduction assembly as recited in claim 1 wherein said buffer is further structured for confronting disposition with an exterior portion of the pericardial bag corresponding to the entry site.

3. An introduction assembly as recited in claim 2 wherein said buffer is disposed on said sheath at a location to at least partially define said predetermined intracardiac length.

4. An introduction assembly as recited in claim 1 further comprising a valve assembly connected to a proximal end of said sheath in flow restricting relation to said central lumen.

5. An introduction assembly as recited in claim 4 wherein said valve assembly is operatively structured to restrict fluid flow through said proximal end at least during operative positioning of the insertion and removal of dedicated instrumentation.

6. An introduction assembly as recited in claim 1 wherein said closure assembly comprises a first segment and a second segment respectively and concurrently disposable interiorly and exteriorly of the entry site and in substantially closing relation thereto.

7. An introduction assembly as recited in claim 6 wherein said operative position of said closure assembly comprises said first and second segments respectively disposed in confronting relation to the entry site on an interior and exterior of the targeted atrium.

8. An introduction assembly as recited in claim 1 wherein said closure assembly is disposable in a substantially collapsed orientation of sufficient dimension to pass through said central lumen of said sheath to the entry site.

9. An introduction assembly as recited in claim 8 wherein said closure assembly is disposable into and expanded orientation of sufficient dimension to assume said operative orientation relative to the entry site.

10. An introduction assembly as recited in claim 9 wherein said closure assembly comprises a first segment and a second segment respectively and concurrently disposable interiorly and exteriorly of the entry site; each of said first and second segments disposable into said compressible and expandable orientations.

11. An introduction assembly as recited in claim 10 wherein each of said first and second segments includes an inherently biased construction facilitating disposition thereof into said expanded orientation and said operative position, upon an exiting thereof from said sheath.

12. An introduction assembly as recited in claim 1 further comprising a stabilizing fixture assembly disposable in interconnecting relation between said sheath and an adjacent portion of the thoracic passage.

13. An introduction assembly as recited in claim 12 wherein said fixture includes an angle compensating structure operative to accommodate an angular orientation of said sheath relative to the thoracic passage.

14. An introduction assembly as recited in claim 12 wherein said stabilizing fixture assembly is structured for removable attachment to a skin surface disposed adjacent the therasic passage.

15. An introduction assembly as recited in claim 1 further comprising a lancet assembly at least partially formed of a flexible material and cooperatively dimensioned with said sheath to facilitate passage therethrough into operative engagement with the targeted atrium.

16. An introduction assembly as recited in claim 15 wherein said lancet comprises a blade disposed at the distal end thereof, said blade selectively disposed between a retracted position and an extended position.

17. An introduction assembly as recited in claim 16 wherein said lancet assembly further comprises an activating structure operative from a proximal end of said lancet assembly and structured to selectively dispose said blade between said retracted and expanded positions.

18. An introduction assembly as recited in claim 1 further comprising a securing assembly disposed and structured to removably secure said buffer on an exterior of the targeted atrium.

19. An introduction assembly as recited in claim 18 wherein said securing assembly comprises a vacuum source, said buffer connected in fluid communication with said vacuum source and structured to exert a securing negative pressure force on the exterior of the pericardial bag.

20. An introduction assembly as recited in claim 19 further comprising a fluid connection disposed at least partially on said sheath in fluid communication between said vacuum source and said buffer.

21. An introduction assembly as recited in claim 19 wherein said buffer includes a collapsible construction.

22. An introduction assembly as recited in claim 19 wherein said buffer comprises a substantially annular configuration connected to an exterior of said sheath and extendable transversely outward therefrom into movement restricting engagement with the exterior of the pericardial bag.

23. An introduction assembly as recited in claim 19 wherein said buffer comprises a plurality of pads connected to an exterior of said sheath and extendable transversely outward therefrom into interruptive, movement restricting engagement with the exterior of the pericardial bag.

24. A method of introducing medical instrumentation through a thoracic passage and into a targeted atrium of the heart, the method comprising:
forming at least one entry site into the target atrium,
positioning a distal end of an introductory sheath through the thoracic passage and the entry site and structuring the distal end to include a predetermined intracardiac length,
passing instrumentation dedicated to perform predetermined cardiac maneuvers along an interior of the introductory sheath and into the targeted atrium through the entry site,
removing the dedicated instrumentation from the targeted atrium through the introductory sheath subsequently to performing the cardiac maneuvers,
positioning a closure assembly through the introductory sheath and into closing relation to the entry site, and
removing the introductory sheath from the thoracic passage.

25. A method as recited in claim 24 comprising respectively positioning a first closure segment and a second closure segment of the closure assembly in confronting relation to interior and exterior portions of the entry site to define the closing relation of the closure assembly.

26. A method as recited in claim 25 passing each of the first and second closure segments through the introductory sheath in a collapsed orientation and disposing corresponding ones of the first and second closures segments into an expanded orientation on an interior and an exterior of the targeted atrium.

27. A method as recited in claim 25 further comprising interconnecting the two closure segments in closing, sandwiching relation to the entry site.

28. A method as recited in claim 24 comprising forming a plurality of entry sites into the targeted atrium; positioning different introductory sheaths through different thoracic passages and through different ones of the plurality of entry sites; structuring the corresponding distal ends of the plurality of introductory sheaths to include predetermined intracardiac lengths; passing instrumentation dedicated to perform predetermined cardiac maneuvers through the plurality of introductory sheaths into the targeted atrium through the corresponding entry sites; removing the dedicated instrumentation from the targeted atrium through corresponding ones of the introductory sheaths upon completion of the intended cardiac maneuvers; positioning different closure assemblies through each of the plurality of introductory sheaths and into closing relation to corresponding ones of the entry sites and removing the plurality of introductory sheaths from the corresponding thoracic passages.

29. A method as recited in claim 28 comprising concurrently disposing at least some the dedicated instrumentation through corresponding ones of the entry sites and into the targeted atrium.
